(19) United States
(12) Patent Application Publication
(10) Pub. No.: US 2024/0023744 A1
(43) Pub. Date: Jan. 25, 2024

(54) NAPPY APPLICATOR

(71) Applicant: Brendan ALLEN, Morayfield (AU)

(72) Inventor: Brendan ALLEN, Morayfield (AU)

(21) Appl. No.: 18/341,945

(22) Filed: Jun. 27, 2023

(30) Foreign Application Priority Data
Jul. 22, 2022 (AU) 2022902053

(51) Int. Cl. A47G 25/90; A61F 13/84

(52) U.S. Cl. CPC A47G 25/90; A61F 13/84; A61F 2013/8476

(57) ABSTRACT

An applicator is described for applying a nappy of the type having a rear panel region joined to a front panel region, and two side fastenings at each side for attaching the front panel to the rear panel around the waist of the wearer, the applicator including: a generally planar portion which is provided with retaining means for temporarily retaining the rear panel of the nappy to the applicator.

### TECHNICAL FIELD

The present invention relates to an applicator for applying a nappy (otherwise known as a diaper) to a baby or young child. The invention has particular application to applying nappies of a commonly used type which have a rear panel region joined to a front panel region, and two side fastenings at each side for attaching the front panel to the rear panel around the waist of the wearer.

### BACKGROUND TO THE INVENTION

Parents or carers of babies and your children are familiar with the need to put a nappy on the baby or child at various times throughout a day, and after washing or bathing. Fitting a nappy is often done using a changing table or other flat surface which is set at about waist height to an adult standing next to the table. The child is placed on their back. Then, holding a new nappy open in one hand, the adult will then lift the child by the feet with their other free hand, and slide the new nappy underneath the child. The fasteners at each side of the nappy then need to be located and then fastened around the waist of the baby. This procedure is often complicated if the child decides to or struggle or resist the efforts of the adult to fit the nappy.

There remains a need for improved techniques to apply nappies to babies and your children.

### SUMMARY OF THE INVENTION

In a first aspect the present invention provides an applicator for applying a nappy of the type having a rear panel region joined to a front panel region, and two side fastenings at each side for attaching the front panel to the rear panel around the waist of the wearer, the applicator including: a generally planar portion which is provided with retaining means for temporarily retaining the rear panel of the nappy to the applicator.

The planar portion may be generally elongate and the retaining means may be provided at either end of the planar portion.

The retaining means may include regions of a hook fastener material.

The retaining means may include a pair of clips.

The applicator may further include a handle portion. The handle portion may be foldably connected to the planar portion.

### BRIEF DESCRIPTION OF THE DRAWINGS

An embodiment of the present invention will now be described, by way of example only, with reference to the accompanying drawings, in which:

FIG. 1 is a perspective view of an applicator for applying a nappy;

FIG. 2 is a plan view of the applicator of FIG. 1;

FIG. 3 is a side view of the applicator of FIG. 1;

FIG. 4 shows a nappy for use with the applicator; and

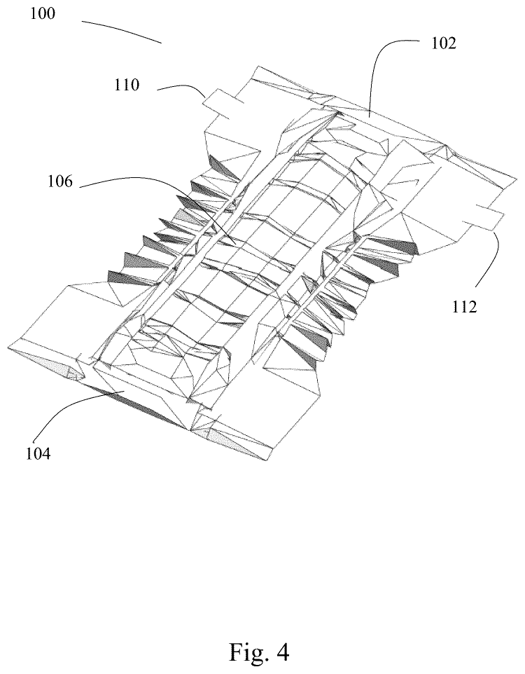

FIG. 5 shows the nappy of FIG. 4 in association with the applicator ready for use.

### DETAILED DESCRIPTION OF THE PREFERRED EMBODIMENT

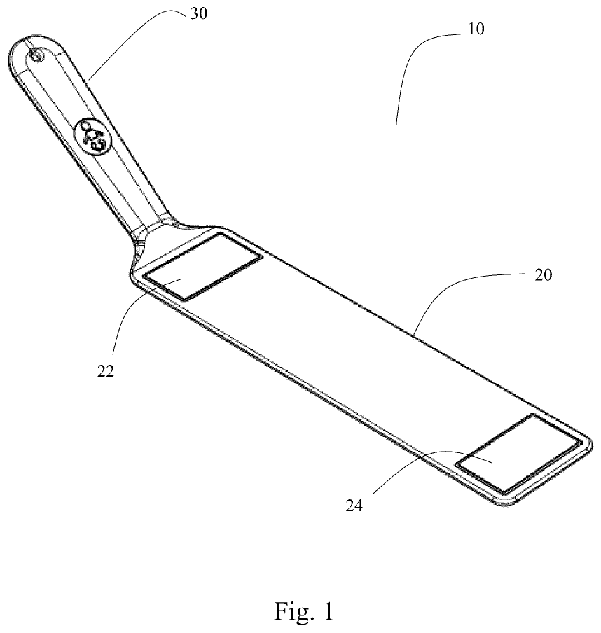

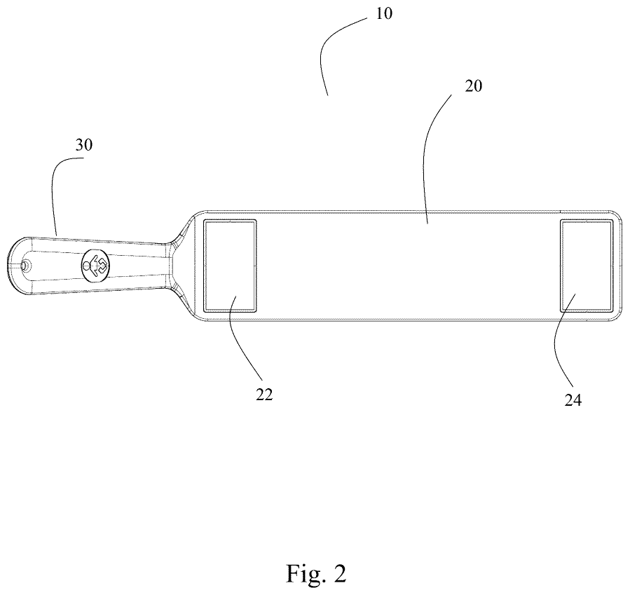

Referring to FIGS. 1 to 3, an applicator 10 for applying a nappy is shown and includes a generally elongate planar body 20 which is provided with two retaining means in the form of regions of hook fastener material 22, 24 which are spaced apart and located at either end of the body. A handle 30 is affixed to the body 20 at an angle of 37 degrees from the plane of the body.

The handle 30 and body 20 are formed in one piece from moulded plastic. The regions of hook material 22, 24 are adhered to the surface of the body.

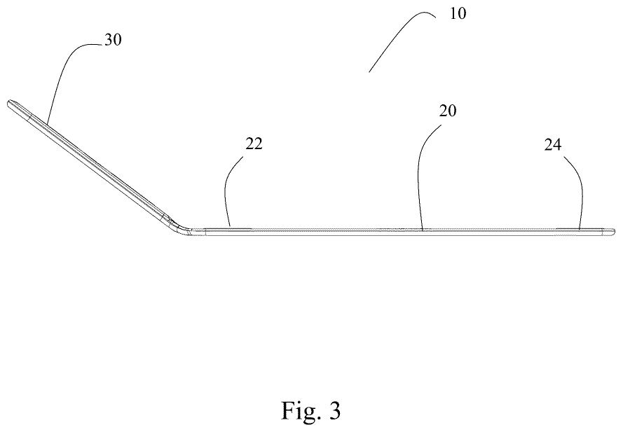

The applicator is intended for use with the type of disposable nappy shown in an opened configuration in FIG. 3. Nappy 100 has a rear panel region 102 joined to a front panel region 104 and a region of absorbent material 106. Two side fastenings in the form of self adhesive tabs 110, 112 are provided at each side of the rear panel for attaching the front panel to the rear panel around the waist of the child in a well known fashion.

To use the applicator top change or apply a nappy, the following method steps are carried out:

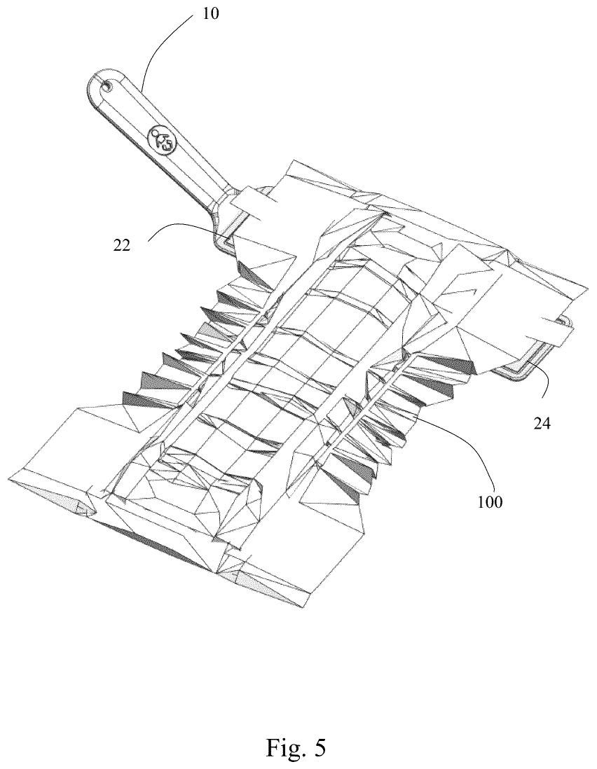

1. As shown in FIG. 5, prepare the applicator by opening out a nappy 100 to be applied and affixing the nappy to applicator 10 by pressing the outside of the waistband region of the rear panel of the nappy against the regions of hook material 22, 24.
2. With the child on the change table, lift the feet of the child to raise their bottom up off the change table surface.
3. Slide the combination of the applicator 10 and the nappy 100 underneath the child to bring the rear waistband portion of the nappy aligned with the child's waist region.
4. Lower the child onto the nappy.
5. Locate the side fastening regions 110, 112 at the ends of the rear waistband portion and pull them around to fasten with the front panel of the nappy. This movement also separates the nappy from engagement with the hook regions of material.
6. Lift child from the change table and retrieve the applicator from underneath the child for subsequent use.

The applicator enables a person to swiftly and accurately position the nappy under the child using one hand. The rear of the nappy is held open, allowing easy grasping of the fastening tabs at the sides of the nappy. The process of applying the nappy is made quicker and more efficient, and improves comfort for the child during a nappy change.

In other embodiments, the applicator may be formed from a wooden material such as bamboo.

In the embodiments described above, the retaining means was provided at either and of the planar portion. In other embodiments it may provided as a continuous region along the planar portion. It may take the form of a single region of hook material.

In some other embodiments, the hook material may be replaced with clips which temporarily hold the rear portion of the nappy to the applicator. In some embodiments a single elongate clip may be used which extends along the planar portion.

In some embodiments the handle of the applicator may be foldably attached to the body to allow for folding of the applicator for compact storage.

Any reference to prior art contained herein is not to be taken as an admission that the information is common general knowledge, unless otherwise indicated.

Finally, it is to be appreciated that various alterations or additions may be made to the parts previously described without departing from the spirit or ambit of the present invention.

1. An applicator for applying a nappy of the type having a rear panel region joined to a front panel region, and two side fastenings at each side for attaching the front panel to the rear panel around the waist of the wearer, the applicator including:
a generally planar portion which is provided with retaining means for temporarily retaining the rear panel of the nappy to the applicator.

2. The applicator according to claim 1 wherein the planar portion is generally elongate and the retaining means is provided at either end of the planar portion.

3. The applicator according to claim 1 wherein the retaining means includes one or more regions of a hook fastener material.

4. The applicator according to claim 1 wherein the retaining means includes at least one clip.

5. The applicator according to claim 1 wherein the retaining means includes a pair of clips.

6. The applicator according to claim 1 further including a handle portion.

7. The applicator according to claim 6 wherein the handle portion is foldably connected to the planar portion.
